(19) United States
(12) Patent Application Publication
(10) Pub. No.: US 2023/0058167 A1
(43) Pub. Date: Feb. 23, 2023

(54) SPORTS EYEWEAR PROTECTOR

(71) Applicant: GOGGLETECHNOLOGY LTD, Dumfries and Galloway (GB)

(72) Inventor: Nathan Dessimone, Dumfries and Galloway (GB)

(21) Appl. No.: 17/950,408

(22) Filed: Sep. 22, 2022

(30) Foreign Application Priority Data
Aug. 16, 2021 (GB) 2111741.1

(51) Int. Cl. A61F 9/02

(52) U.S. Cl. CPC A61F 9/025

(57) ABSTRACT

A sports eyewear lens protector 10 for temporarily protecting the sports eyewear lens, the sports eyewear lens protector comprising: at least one disposable protector layer comprising an optically clear film formed from a compostable polymer material, the protector layer include a lens protecting portion 14 for covering at least a portion of the lens of the eyewear, a handle portion 16 extending from a first side of the lens protecting portion for allowing a user of the eyewear to grip and remove the protector layer from the eyewear, and at least two mounting apertures 20a,20b for mounting the lens protector to the sports eyewear lens, each mounting aperture being disposed to a side of the lens protecting portion.

### CROSS REFERENCE TO RELATED APPLICATIONS

This application claims the benefit of U.K. Patent Application No. GB2111741.1, filed on Aug. 16, 2021. The entire disclosure of the above application is incorporated herein by reference.

### FIELD OF INVENTION

The present invention relates to sports eyewear protectors and particularly but not exclusively to motorsports eyewear protectors.

### BACKGROUND TO THE INVENTION

Lenses for eyewear, especially protective eyewear such as goggles used in sports and outdoor activities, are prone to scratching, damage and/or fouling from debris which can obscure or distort the vision of the user. During sporting activities, for example motocross, this can be a safety concern as unobstructed vision helps to avoid dangers and reduce the risk of accidents which can cause serious injury to the user and/or others. Furthermore, replacing damaged eyewear, or in some cases the damaged eyewear lenses, is often costly.

Various forms of protective films for eyewear have been created to address the issues with scratching, damage and/or fouling.

One solution, commonly known as tear-offs, is provided by a stack of protective films made from a polymer which are placed over the lens of the eyewear. Each individual film includes a section which fits over at least a portion of the eyewear lens and a strip extending outwards which serves as a handle. When the film gets soiled or damaged the user reaches up grips the handle and pulls the individual film away from the stack. The soiled or damaged protective films are discarded. The discarded films litter the environment which is especially problematic because the material is not naturally biodegradable.

A further solution, commonly known as roll-offs, is provided by a clear protective film made from a polymer stored in a rolled configuration within a storage container. The storage container is disposed to one side of the eyewear and the clear protective film extends from the storage container to the other side of the eyewear, covering a portion of the eyewear lens. When the portion of the clear protective film disposed to the eyewear lens has been damaged or soil the user advances the roll so as to locate a fresh portion of the clear protective film in the appropriate position. The soiled or damaged portion of the protective film is stored in a further container disposed on the opposite side. The further container provides a convenient way to store the used roll, however, it still needs to be disposed of later.

It is an object of the present invention to reduce or substantially obviate the aforementioned problems.

### STATEMENT OF INVENTION

According to a first aspect of the present invention there is provided a sports eyewear lens protector for temporarily protecting the sports eyewear lens, the sports eyewear lens protector comprising at least one disposable protector layer. The disposable protector layer being an optically clear film comprised of or is formed from a compostable polymer material. The protector layer includes a lens protecting portion for covering at least a portion of the lens of the eyewear, a handle portion extending from a first side of the lens protecting portion for allowing a user of the eyewear to grip and remove the protector layer from the eyewear, and at least two mounting apertures for mounting the lens protector to the sports eyewear lens, each mounting aperture being disposed to a side of the lens protecting portion.

Temporary protective film layers provide a speedy and effective way to maintain or restore visibility. The use of a compostable optically clear film for temporary protective layers, e.g. tear-off protectors, allow the user to simply discard the protective layer without worrying about the impact on the environment. This will help reduce or address the environmental concerns of the user, sports organiser or other authorities.

The sports eyewear protector may be retrofitted to the sports eyewear lens. The sports eyewear protector may be considered a tear-off protector or a temporary protector or a disposable protector. The protector layer may be considered a tear-off protector layer or a temporary protector layer.

The compostable material may be an aerobic compostable material. The compostable material may be certified according to DIN EN :13432:2000-12, ASTM D6400 and/or other similar certification standards. The compostable material may take up to or no more than 14 weeks to break down entirely, preferably up to or no more than 12 weeks, and more preferably up to or no more than 11 weeks. The compostable material may take between 3 to 8 weeks to substantially break down.

Compostable material, especially materials which decompose aerobically, allows the sports eyewear protector to biodegrade naturally.

The compostable material may comprise cellulose. The cellulose may preferably be cellulose fibres, such as those obtained from wood. The material may be an acetate ester of cellulose. The material may be a cellulose diacetate.

The use of cellulose is advantageous as it uses natural renewable raw materials as well as producing a compostable and biodegradable material.

The material may comprise additives. The additives may include at least one of a plasticizer, antiblocking agent and release agent.

The material may be produced by dissolving wood fibres (di-acetate flakes) in acetone to form a spinning solution. The additives may improve processability of the spinning solution and/or performance of the film.

The thickness of the optically clear film may be between 10 to 600 μm, preferably 14 to 500 μm, more preferably 14 to 115 μm. The thickness of the optically clear film may be up to 115 μm. The thickness of the optically clear film may be about 75 μm.

The thinner films advantageously degrade quicker, with thicknesses of up to 115 μm being home compostable. Thicker films may take longer to degrade, or require further processes, but may provide better levels of protection. A thickness of about 75 μm provides a good balance between compostability and protection.

The material of the optically clear film may have a tensile strength at break in the range of 70 to 120 Nmm⁻², preferably in the range of 80 to 110 Nmm⁻². The tensile strength of the material at break may be according to ASTM D 882.

The material of the optically clear film may have an elongation at break in the range of 25 to 50%, preferably 25 to 45%. The elongation at break may be according to ASTM D 882.

The material of the optically clear film may have an E-modules in the range of 1700-2500 N/mm², preferably 2000-2500. The E-modules may be according to ASTM D 882.

The mechanical properties of the material, for example the tensile strength, are advantageous for a protector layer because it reduces the likelihood of breaks when the user removes said protector layer from the eyewear.

The protector layer may be hydrophobic.

A hydrophobic coating may be applied to a surface of the protector layer. That is to say that the hydrophobic coating may be applied to a surface which is not in contact with the sport eyewear lens during use.

The protective layer may have a water contact angle greater than 90%. The material of the optically clear film may provide a water contact angle greater than 90%. Water contact angles greater than 90% provide hydrophobic properties.

The material of the optically clear film may have a surface tension between 34-42 mN/m. The optically clear film may have a surface tension between 38-42 mN/m. The surface tension may be according to ISO 8296 DIN 53364. The surface tension is measured on the day of production, values may decrease in time.

The material of the optically clear film may have a dynamic friction coefficient between 04 to 0.6.

The optically clear film may have a haze in the range of 0.6% and 6,1%, preferably between 0.6% and 1.7%, more preferably about 1.4%. The haze may be according to ASTM D 1003.

The material of the optically clear film may have a transmittance of about 93% or at least 93%. The transmittance may be according to ASTM D 1003.

The optically clear film may have a refractive index in the range of 1.485 to 1.490, preferably 1.485.

The optically clear film may have anti-fog properties.

The material of the optically clear film may have a water vapour transmission rate in the range of 122.2 to 2283 g/m²/day at 25° C. and 75% relative humidity. The water vapour transmission rate may be about 443 g/m²/day at 25° C. and 75% relative humidity. The water vapour transmission rate provides good anti-fog properties.

The material of the optically clear film may have a water vapour transmission rate in the range of 254 to 1890 g/m²/day at 38° C. and 90% relative humidity. Preferably the water vapour transmission rate may be about 761 g/m²/day at 38° C. and 90% relative humidity. Preferably, the water vapour transmission rate may be about 850 g/m²/day at 23° C. and 0% relative humidity

According to ASTM D543-87, the material of the optically clear film may have low resistance to Ketones; be attacked by esters, moderate to concentrated strong acid and bases, as well as be resistant to non-polar solvents.

An adhesive may be disposed on a surface of the lens protecting portion for detachably attaching the protector layer to the sport eyewear lens or a surface of a same or similar protector layer. The adhesive may be a dry mount adhesive. The adhesive may be self-wetting.

The adhesive may comprise a silicone adhesive, preferably a pressure sensitive silicone adhesive.

The adhesive may be an optically clear adhesive. The adhesive may have the same or similar refractive index as the optically clear film. The refractive index of the adhesive may substantially match the refractive index of the optically clear film.

The refractive index of the optically clear film may substantially match or be similar to the refractive index of the eyewear lens.

The refractive indices of the optically clear film and adhesive may match or be substantially similar to the refractive index of the sport eyewear lens.

Matching or having similar or substantially similar refractive indices between the different components, such as the adhesive, optically clear film and lens, limits perceptible internal reflections which improves vision through the sports eyewear protector.

A first liner may be detachably attached to a surface of the lens protecting portion of the optically clear film. The first liner may provide a means to temporarily cover the adhesive.

A second liner may be detachably attached to a surface of the lens protecting portion of the optically clear film. The second liner provides a means to temporarily cover a front surface of the protective layer which provides protection during transportation and/or fitment.

The lens protecting portion of the optically clear film may be sized and shaped to substantially correspond to the lens of the eyewear. That is to say that the lens protecting portion may substantially follow a perimeter of the frame disposed around the lens.

A gripping aperture may be provided in the handle portion. The aperture may be sized to receive a portion of a gloved finger or it may be sized to allow a gloved hand to pinch through the aperture. This aperture allows the user to easily remove the protective layer.

A mounting aperture may be provided in the handle portion. The mounting aperture may be suitable for receiving a protrusion, catch, stub or similar on the sport eyewear lens. The mounting aperture in the handle portion allows the handle to be substantially folded which ensures that it does not significantly flap in the wind when fitted.

A hard scratch resistant coating may be applied to a second surface of the protective layer. The hard scratch resistant coating may have a hardness rating of 7H or 9H.

The protective layer may be flexible.

A plurality of protector layers may be provided. Each protector layer may be detachably attached to at least one other protector layer to form a stack of protector layers. The lens protection portion of each protector layer may be detachably attached while the handle portions are not.

Each protector layer may be detachably attached using an adhesive. The adhesive may be an optically clear adhesive. The refractive index of the optically clear adhesive may match or be substantially similar to that of the protector layers and the eyewear lens. The handle portions of each protector layer may not be detachably attached

A preformed stack of protector layers reduces the time required to fit the sports eyewear lens protector to the sport eyewear lens.

A kit of parts for forming a sports eyewear lens protector may be provided. The kit of parts may comprise a plurality of protective layers.

Each protective layer may have the same or substantially similar refractive index.

An Indicia display region may be provided. An indicia display region may be provided in the lens protecting portion, preferably the indicia display region may be provided in close proximity to a peripheral edge of the protector layer. The indicia display region may display a symbol such as an image, trademark, flag or similar.

A marker may be provided in the handle portion. The marker may provide indication of the type of protective layer, for example the marker may be a green pattern which indicates that the protective layer is compostable. The marker may also be a type of indicia, such as a symbol, flag or image.

According to a second aspect of the present invention there is provided a sports eyewear lens protector for retrofitting to a sporting eyewear lens, the sports eyewear lens protector comprises an optically clear flexible laminate film for fitment to a lens of sporting eyewear, the laminate film comprising a scratch resistant layer and a support layer attached to a bottom surface of the scratch resistant layer, and an optically clear silicone adhesive for removably adhering the flexible laminate film to the lens of the sporting eyewear, the optically clear silicone adhesive being applied to a bottom surface of the support layer.

The sports eyewear lens protector of the second aspect is semi-permanent in the sense that it is intended to be attached and used for months to years before removal. The semi-permanent lens protector may adhere to the sport eyewear lens for at least 24 months without damaging or leaving a residue on the eyewear lens when removed. The lens protector can easily be cleaned and wiped down while the scratch resistant layer prevents damage to the sports eyewear lens. The lens protector may be considered a lens cover or a partial lens cover. Compared to tear-offs or roll-offs, this reduces the amount of waste. The sports eyewear lens protector is therefore environmentally friendly.

The sports eyewear lens protection may be detachably attached to the sports eyewear lens, in use.

The scratch resistant layer may be a coating applied to the support layer. That is to say the scratch resistant layer may be a scratch resistant coating layer. The scratch resistant layer may be a hard coating. The hardness of the scratch resistant coating may be 7H, 8H or 9H. Preferably, the hardness of the scratch resistant coating will be 9H. The hardness may be determined by JIS K 56000. The hardness may be determined using the Wolf Wilborn test. The higher the hardness rating the greater the degree of protection.

The scratch resistant coating may be a UV-curable hard coat.

The scratch resistant layer has a thickness of between 10 to 40 μm. Preferably the thickness of the scratch resistant layer may be between 18 to 38 μm. The thickness of the scratch resistant layer may be about 38 μm. The thickness of the scratch resistant layer may be about 18 μm.

The support layer may be polyethylene terephthalate (PET). The thickness of the support layer may be between 100 and 200 μm. The thickness of the support layer may be about 188 μm.

The support layer is between 11 to 4 times as thick as the scratch resistant layer, preferably about 10.44 times as thick, more preferably 4.95 times as thick.

The optically clear silicone adhesive may be a pressure sensitive adhesive. The optically clear silicone adhesive may be applied in a thickness of between 30 to 50 μm.

The optically clear silicone adhesive may be UV resistant. The provision of a UV resistant silicone adhesive prevents or limits discolouration of the adhesive which would negatively impact the user.

The optically clear silicone adhesive may have the same properties as that discussed above. For example, it may have the same or similar refractive index as the optically clear flexible laminate film.

The refractive index of the sports eyewear lens protection may substantially match or be similar to the refractive index of the sports eyewear lens.

Matching or having similar or substantially similar refractive indices between the different components, such as the adhesive, optically clear laminate film and lens, limits perceptible internal reflections which improves vision through the sports eyewear protector.

The scratch resistant layer may be hydrophobic. A hydrophobic coating may be applied to a top surface of the scratch resistant layer. The water contact angle of the scratch resistant layer may be greater than 90°.

The hydrophobic coating may have a surface with microscale roughness and/or a nanoscale roughness formed thereon.

The hydrophobic coating may be a curable hydrophobic composition for application to a surface. The surface may be a surface of the scratch resistant layer. The curable hydrophobic coating may form a surface having a microscale roughness and/or the nanoscale roughness once cured.

The hydrophobic composition may include nanoparticles or precursors capable of forming nanoparticles. The composition may include microparticles. The composition may include an organic solvent.

Transparency of the optically clear flexible laminate may be between 88 to 96%, preferably 90 to 93%. The transparency of the optically clear flexible laminate may be determined by JIS K 7105.

Haze of the optically clear flexible laminate may be between 1 to 1.2%. The haze may be determined by JIS K7105.

The optically clear silicone adhesive may have an adhesion strength of between 3 to 10 gf/25 mm. The adhesion strength may be 7±2 gf/25 mm or 5±2 gf/25 mm. The adhesion strength may be determined by JIS Z 0237.

The composition of the optically clear silicone adhesive may include silanol terminated polydiorganosiloxane, preferably 100 parts by weight. The silanol terminated polydiorganosiloxane may have a viscosity of 100 to 200000 cSt at 25° C. The composition may include an aminoxy group-containing organosilicon compound, preferably 0.1 to 30 parts by weight. The aminoxy group-containing organosilicon compound may have on average more than two organoaminoxy groups per molecule. The composition may include an inorganic filler, preferably 5 to 300 parts by weight. The composition may include an oligomer contain acryl groups, preferably 1 to 30 parts by weight.

The optically clear flexible laminate film is shaped to fit within a frame holding the lens of the sporting eyewear. Preferably, the optically clear flexible laminate film, when fitted to the sports eyewear lens, covers a portion of the sports eyewear lens within the frame holding the sporting eyewear lens. That is to say, that there are portions of the sports eyewear lens not covered by the optically clear flexible laminate film, i.e. it partially covers the sports eyewear lens in use, preferably a central portion of the sports eyewear lens. Preferably, the film is spaced away from the frame. The sports eyewear lens protector may be considered a universal fit. That is to say, that it may be sized and shaped so as to fit within any of the currently available sporting eyewear frames.

The sports eyewear lens protector may be substantially planar before fitting to the sports eyewear lens. When fitted, the sports eyewear lens protector may follows the curvature of the sports eyewear lens. A first liner may be detachably attached to an outer surface of the sports eyewear lens protector. The first liner may be a temporary liner which is used to protect the lens protector prior to use.

A second liner may be detachably attached to a second surface of the lens protecting portion of the optically clear film, the second surface being opposite the first surface. The second liner provides a means to temporarily cover the front surface which provides protection to the protective layer before fitment to the lens.

The term optically clear should be considered to refer to the material having no perceptible visible distortion or scatter. Optically clear may also mean transparent. The film may be colourless or may have a hue, shade or tint.

The sports eyewear may be motorsport goggles, masks, helmets or similar. The sports eyewear may also be those used in other sports, such as skiing or similar.

### BRIEF DESCRIPTION OF THE DRAWINGS

For a better understanding of the present invention, and to show more clearly how it may be carried into effect, reference will now be made by way of example only to the accompanying drawings, in which:

FIG. 1 shows a view of a sports eyewear protector according to a first aspect of the present invention;

FIG. 2 shows a view of a sports eyewear protector according to the first aspect of the present invention with a stack of protector layers;

FIG. 3 shows a view of a sports eyewear protector according to a second aspect of the present invention;

FIG. 4 shows a cross sectional view of the sports eyewear protector of FIG. 3; and

FIG. 5 shows a view of the sports eyewear protector of FIG. 3 applied to sports goggles.

### DESCRIPTION OF PREFERRED EMBODIMENTS

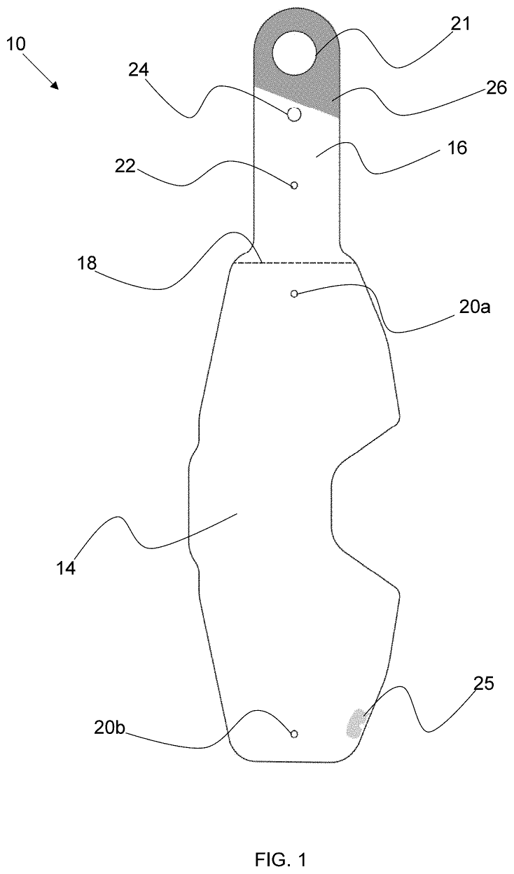

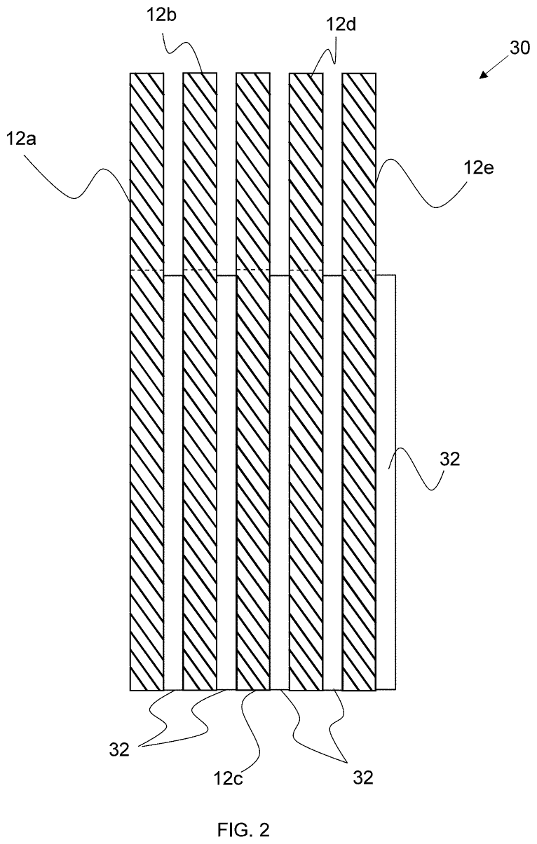

Referring firstly to FIG. 1, a sports eyewear protector according to the first aspect of the present invention is generally indicated at 10. In the embodiment shown in FIG. 1, the sports eyewear protector has a single disposable protector layer 12. However, in other embodiments, such as that shown in FIG. 2, more than one protector layer 12 may be provided.

An optically clear film forms the disposable protector layer. The material of the optically clear film is a compostable material, preferably a compostable polymer material. In the current embodiment, the material of the optically clear film is an acetate ester of cellulose, preferably cellulose diacetate. The cellulose preferably comes from wood fibre or a wood pulp.

In the current embodiment the optically clear film has a thickness of about 75 μm. The clear optical film has haze of 1.1 to 1.4%, preferably 1.4%, transmittance of about 93%, a refractive index of about 1.485, water vapour transmission rate of about 443 g/m²/day at 25° C. and 75% relative humidity, a surface tension of 38-42 mN/m, a tensile strength in the range of 80-110 Nmm⁻², and a dynamic friction coefficient in the range of 0.4-0.6. In other embodiments the thickness of the film may be different and have different properties, examples of these are shown in Table 1 and Table 2 further below.

A lens protecting portion 14 is provided in the disposable protector layer. In the current embodiment, the lens protecting portion 14 is sized to substantially cover a sport eyewear lens. A portion of the peripheral edge of the protective layer is shaped to substantially correspond to the shape of the sports eyewear lens.

A handle portion 16 extends out from a first side of the lens protecting portion 14. The handle may extend by 7 to 11 cm, preferably 8 to 10 cm.

A fold line 18, or a score line, reduced thickness section or similar is provided between the lens protecting portion 14 to the handle portion 16.

In the current embodiment two mounting apertures 20a, 20b are disposed in the lens protecting portion 14, but the number of mounting apertures may correspond to the number of mounting protrusions on the sports eyewear. The mounting apertures 20a, 20b are sized and shaped to correspond to the mounting protrusions. For example, first and second apertures may be spaced apart within the range of 13 to 19 cm, preferably within the range of 14.5 to 16.5 cm and be circular in shape. The layout of the mounting aperture may also correspond to the layout of the mounting protrusions.

The first mounting aperture 20a is disposed to the first side of the lens protecting portion 14 and inwardly spaced from the fold line 18. The second mounting aperture 20b is disposed to a second side of the lens protecting portion 14 and inwardly spaced from the peripheral edge.

A gripping aperture 21 is disposed in the handle portion 16 towards the end distal the lens protecting portion 14. The gripping aperture 21 is sized to receive a gloved finger so as to allow a user of the sports eyewear to remove the protective layer 12.

A first aperture 22 is provided in the handle portion 16. The first aperture 22 has the same size and shape of the mounting apertures 20a, 20b.

A second aperture 24 is provided in the handle portion 16. The second aperture 24 is the same shape but has a larger size than the first aperture 22 and mounting apertures 20a, 20b.

During use the first and second apertures 22, 24 of the handle portion 16 provide a means to secure a folded over handle portion 16. In the folded configuration both the first and second aperture 22, 24 receive a mounting protrusion of the sport eyewear. The folded configuration prevents the handle portion 16 from significantly moving as a result of wind.

A symbol 25 is provided in an indica display region. The symbol 25 is disposed in close proximity to the peripheral edge of the lens protecting portion 14.

A marker 26 is disposed to the distal end of the handle portion 16. The marker surrounds the gripping aperture 22.

A first liner may be provided to cover a front face of the protector layer. The first liner protects the front face of the protector layer before the protector is used.

Adhesive (such as that described below) for affixing the protector layer to the sport eyewear lens or a same or similar protector layer may be provided in some embodiments. The adhesive is provided on a rear face of the protector layer. The adhesive is provided in the lens protecting portion of the protector layer.

A second liner may be provided to cover the rear face of the protector layer. The second liner protects the adhesive and/or the rear face of the protector layer.

Referring to FIG. 2, a sports eyewear protector is generally indicated at 30.

In the current embodiment the sports eyewear protector 30 includes five protector layers 12a-e formed into a stack; however, in other embodiments there may be more or less protector layers. Reference numbers for the same or similar features to those described above will be maintained.

Adhesive 32 is applied to the rear face of each protector layer 12a-e. The adhesive applied to the first, second, third and fourth protector layers 12a-12d adhere the rear face of the respective protector layer to the front face of the next protector layer in the stack. For example, the rear face of the first protector layer 12a is adhered to the front face of the second protector layer 12b by adhesive 32. To mount the sports eyewear protector 30, the rear face of the fifth protector layer 12e is adhered to the sports eyewear lens.

TABLE 1

| | | | | | | | | | | | | |
|---|---|---|---|---|---|---|---|---|---|---|---|---|
| Thickness | 14 | μm | 24 | μm | 30 | μm | 50 | μm | 75 | μm | 95 | μm |
| Yield | 54.11 | m²/kg | 31.57 | m²/kg | 25.25 | m²/kg | 15.15 | m²/kg | 10.10 | m²/kg | 7.97 | m²/kg |

| Tensile | 80-110 Nmm⁻² | | | | | | | | | | | |
|---|---|---|---|---|---|---|---|---|---|---|---|---|
| strength | | | | | | | | | | | | |
| at break | | | | | | | | | | | | |
| Elongation | 25-45% | | | | | | | | | | | |
| at break | | | | | | | | | | | | |
| E-modulus | 2000-2500 N/mm² | | | | | | | | | | | |
| Haze | 0.6% | | 0.7% | | 0.9% | | 1.0% | | 1.4% | | 1.7% | |
| Transmittance | 93% | | | | | | | | | | | |
| Refractive Index | 1.485 | | | | | | | | | | | |
| W.V.T.R at | 2283 | g/m²/day | 1279 | g/m²/day | 1065 | g/m²/day | 639 | g/m²/day | 443 | g/m²/day | 348 | g/m²/day |
| 25° C. & 75% | | | | | | | | | | | | |
| Rel. H | | | | | | | | | | | | |

| | | | | | | | | | | |
|---|---|---|---|---|---|---|---|---|---|---|
| W.V.T.R at | n/a | 1890 | g/m²/day | 1708 | g/m²/day | n/a | 761 | g/m²/day | 254 | g/m²/day |
| 28° C. & 90% | | | | | | | | | | |
| Rel. H | | | | | | | | | | |

| | | | | | | | | |
|---|---|---|---|---|---|---|---|---|
| W.V.T.R at | n/a | n/a | n/a | 880 | g/m²/day | 850 | g/m²/day | n/a |
| 23° C. & 0% | | | | | | | | |
| Rel. H | | | | | | | | |

| | |
|---|---|
| Equilibrium | ±2 |
| moisture | |
| content at | |
| 23° C. & 50% | |
| Rel.H | |
| Surface | 38-42 mN/m |
| tension | |
| Dynamic | 0.4-0.6 |
| Friction | |
| coefficient | |

TABLE 2

| | | | | | | | | | | | | | | |
|---|---|---|---|---|---|---|---|---|---|---|---|---|---|---|
| Thickness | 115 | μm | 150 | μm | 200 | μm | 250 | μm | 300 | μm | 360 | μm | 500 | μm |
| Yield | 6.64 | m²/kg | 5.13 | m²/kg | 3.82 | m²/kg | 3.08 | m²/kg | 2.56 | m²/kg | 2.14 | m²/kg | 1.54 | m²/kg |

| Tensile | 80-110 Nmm⁻² | | | | | | | |
|---|---|---|---|---|---|---|---|---|
| strength | | | | | | | | |
| at break | | | | | | | | |
| Elongation | 30-50% | | | | | | | |
| at break | | | | | | | | |
| E-modulus | 1700-2000 N/mm² | | | | | | | |
| Haze | 1.7% | 1.7% | | 1.9% | 2.6% | 2.6% | 4.9% | 6.1% |
| Transmittance | 93% | | | | | | | |
| Refractive Index | 1.490 | | | | | | | |
| W.V.T.R at | n/a | 124.6 | g/m²/day | n/a | n/a | n/a | n/a | n/a |
| 25° C. & 100% | | | | | | | | |
| Rel. H | | | | | | | | |
| W.V.T.R at | n/a | 122.2 | g/m²/day | n/a | n/a | n/a | n/a | n/a |
| 25° C. & 75% | | | | | | | | |
| Rel. H | | | | | | | | |

| | |
|---|---|
| W.V.T.R at | n/a |
| 23° C. & 0% | |
| Rel. H | |
| Equilibrium | ±2% |
| moisture | |
| content at | |
| 23° C. & 50% | |
| Rel. H | |
| Surface | 34-38 mN/m |
| tension | |
| Dynamic | 0.4-0.6 |
| Friction | |
| coefficient | |

In Tables 1 and 2 Haze and transmittance may be determined using the method in ASTM D 1003. The tensile strength at break, elongation at break and E-modules may be determined using the method in ASTM D 882 standard. The surface tension may be according to ISO 8296 DIN 53364.

In Tables 1 and 2 W.V.T.R stands for water vapour transmission rate, and Rel.H means relative humidity.

In Tables 1 and 2 the thickness, yield, surface tension may be measured at day of material production.

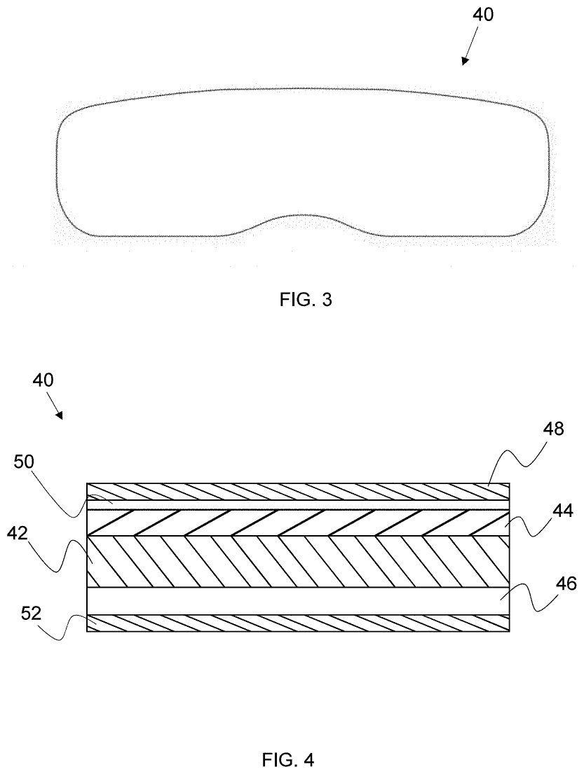

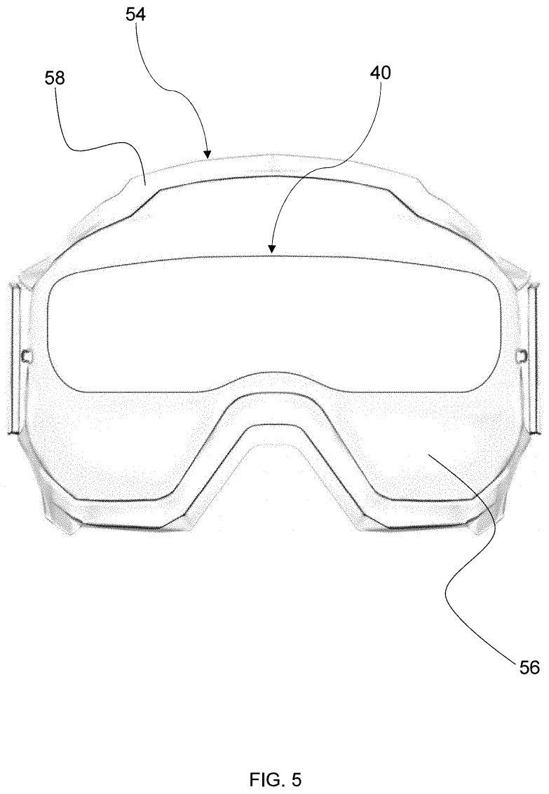

Referring to FIGS. 3 and 4, a sports eyewear protector according to the second aspect of the present invention is generally indicated at 40.

A support layer 42 is made from polyethylene terephthalate (PET). In the current embodiment the support layer has a thickness of about 188 μm.

A hard scratch resistant coating layer 44 is disposed to a first surface of the support layer 42. In the current embodiment, the hard scratch resistant coating 44 is created from a UV curable hard coating. Other forms of hard scratch resistant coatings known to the skilled person may be used.

In the current embodiment the hard scratch resistant coating layer 44 has a thickness of 38 μm. In other embodiments the thickness may differ, for example, the thickness may be 18 μm.

In the current embodiment, the hard scratch resistant coating layer 44 provides a 9H hardness.

The support layer 42 and the scratch resistant layer 44 provide the main protective elements of the sport eyewear protector 40 when fitted to the lens of the sports eyewear. A first surface of the hard scratch resistant coating layer may be considered the outer surface of the sports eyewear protector 40.

Both layers 42, 44 form an optically clear laminate film. The optically clear laminate film is flexible allowing the film to substantially follow a curved sports eyewear lens. In the current embodiment the transparency of the optically clear laminate film is 92% and the haze is 1.2%.

An optically clear silicone adhesive 46 is disposed to a second surface of the support layer 42 opposite the first surface. The optically clear adhesive is a pressure sensitive adhesive.

In the current embodiment the optically clear silicone adhesive layer 46 provides an adhesion strength of 7±2 gf/25 mm and has a thickness of 50 μm. In other embodiments the thickness of the optically clear silicone adhesive layer 46 is 30 μm and provides an adhesion strength of 5±2 gf/25 mm. The thickness of the adhesive may differ once the sports eyewear protector is fitted to the lens.

A first protective liner 48 is attached to a first surface of the hard scratch resistant coating layer 44 by a pressure sensitive silicone adhesive layer 50. The first protective liner 48 can be removed prior to or after fitment of the sports eyewear protector 40 to the sport eyewear lens.

A second protective liner 52 is attached to the optically clear silicone adhesive layer 46. The second protective liner 52 maintains the optically clear silicone adhesive layer 46 prior to fitment of the sports eyewear protector 40 to the sports eyewear lens.

FIG. 5 shows the sport eyewear protector 40 applied to sports goggles 54. There are many shapes and types of sports goggles but generally they include a lens 56 and a frame 58 for holding the lens 56. The lens 56 is typically curved and is made from a clear polycarbonate. The frame 58 provides a perimeter to the lens 56.

In use, the goggles 54 sit on a portion of the user's face and the lens 56 is disposed to the user's eyes. The lens 56 is spaced away from the face by the frame 58 and is typically sized to not overly limit the user's perception.

To fit the sports eyewear protector 40, the second protective liner 52 is removed. The sports eyewear protector 40 is then aligned with the lens 56, preferably centrally so that it will coincide with the eyes of the user when the goggles 54 are worn. Once aligned correctly, the sports eyewear protector 40 is disposed to the lens 56 which causes the optically clear laminate film to adhere to the lens 56 through the optically clear silicone adhesive 46. The first protective liner 48 may be removed at the same time as the second protective liner 52 or once the sports eyewear protector 40 is adhered to the lens 56.

In the current embodiment, the sport eyewear protector 40 is sized and shaped so that it fits to a range of different sports eyewear brands.

The embodiments described above are provided by way of example only, and various changes and modifications will be apparent to persons skilled in the art without departing from the scope of the present invention as defined by the appended claims.

1. A sports eyewear lens protector for retrofitting to a sports eyewear lens comprising:
an optically clear flexible laminate film for fitment to the sports eyewear lens, the laminate film comprising a scratch resistant layer and a support layer attached to a bottom surface of the scratch resistant layer; and
an optically clear silicone adhesive for removably adhering the flexible laminate film to the sports eyewear lens, the optically clear silicone adhesive being applied to a bottom surface of the support layer.

2. The sports eyewear lens protector of claim 1, wherein the scratch resistant layer is a coating applied to the support layer.

3. The sports eyewear lens protector of claim 1, wherein the scratch resistant layer is a hard coating with a hardness rating of 7H, 8H or 9H.

4. The sports eyewear lens protector of claim 1, wherein the scratch resistant layer has a thickness of about 18 to 38 μm.

5. The sports eyewear lens protector of claim 1, wherein the support layer comprises polyethylene terephthalate.

6. The sports eyewear lens protector of claim 1, wherein the support layer has a thickness of about 188 μm.

7. The sports eyewear lens protector of claim 1, wherein the optically clear silicone adhesive is pressure sensitive.

8. The sports eyewear lens protector of claim 1, wherein the scratch resistant layer is hydrophobic.

9. The sport eyewear lens protector of claim 1, wherein the optically clear silicone adhesive is UV resistant.

10. The sports eyewear lens protector of claim 1, wherein the optically clear flexible laminate film has a transparency of 89 to 93%.

11. The sports eyewear lens protector of claim 1, wherein the optically clear flexible laminate film has a haze between 1 to 1.2%.

12. The sports eyewear lens protector of claim 1, wherein the sports eyewear lens protector is a semi-permanent sport eyewear lens protector.

13. A sports eyewear lens protector for retrofitting to a sports eyewear lens comprising:
an optically clear flexible laminate film for fitment to the sports eyewear lens, the laminate film comprising a scratch resistant hydrophobic layer and a support layer attached to a bottom surface of the scratch resistant layer; and
an optically clear silicone adhesive for removably adhering the flexible laminate film to the sports eyewear lens, the optically clear silicone adhesive being applied to a bottom surface of the support layer.

14. The sports eyewear lens protector of claim 13, wherein scratch resistant hydrophobic layer may have a surface with at least a microscale roughness formed thereon.

15. The sports eyewear lens protector of claim 13, wherein the scratch resistant hydrophobic layer may be greater than 90°.

16. A sports eyewear lens protector for temporarily protecting the sports eyewear lens, the sports eyewear lens protector comprising:
at least one disposable protector layer comprising a lens protecting portion for covering at least a portion of the lens of the eyewear, a handle portion extending from a first side of the lens protecting portion for allowing a user of the eyewear to grip and remove the protector layer from the eyewear, and at least two mounting apertures for mounting the lens protector to the sports eyewear lens, in which each mounting aperture is disposed to a side of the lens protecting portion, wherein the disposable protector layer is an optically clear film formed from a compostable material.

17. The sports eyewear lens protector of claim 16, wherein the compostable material is an acetate ester of cellulose.

18. The sports eyewear lens protector of claim 16, wherein the optically clear film has a thickness between 14-500 μm.

19. The sports eyewear lens protector of claim 16, wherein the protector layer is hydrophobic.

20. The sports eyewear lens protector of claim 16, wherein the optically clear film has a surface tension between 34-42 mN/m, preferably 38-42 mN/m.
